(19) United States
(12) Patent Application Publication
(10) Pub. No.: US 2006/0063977 A1
(43) Pub. Date: Mar. 23, 2006

(54) Adjustable retractor support

(75) Inventors: Todd W. Sharratt, Stillwater, MN (US); William Ryder, Victoria, MN (US); Steven M. LeVahn, St. Paul, MN (US)

Correspondence Address: WESTMAN CHAMPLIN & KELLY, P.A., SUITE 1400 - INTERNATIONAL CENTRE, 900 SECOND AVENUE SOUTH, MINNEAPOLIS, MN, 55402-3319, US

(73) Assignee: The LeVahn Intellectual Property Holding Company, LLC, St. Paul, MN (US)

(21) Appl. No.: 11/274,776

(22) Filed: Nov. 15, 2005

Related U.S. Application Data
(63) Continuation-in-part of application No. 10/684,764, filed on Oct. 14, 2003.
Application No. 10/684,764 is a division of application No. 09/624,950, filed on Jul. 25, 2000, now Pat. No. 6,663,563.
Application No. 09/624,950 is a continuation-in-part of application No. 09/409,294, filed on Sep. 29, 1999, now Pat. No. 6,210,325.
(60) Provisional application No. 60/102,788, filed on Oct. 2, 1998.

(51) Int. Cl. A61B 1/32

(52) U.S. Cl. 600/212

(57) ABSTRACT

A retractor system includes an adjustable arm having a proximal end and a distal end where a cable is disposed through the adjustable arm from the proximal end to the distal end. A housing is attached to a proximal end of the adjustable arm. The proximal end has a carriage movably disposed therein that engages one end of the cable. A tensioning mechanism is positioned within the housing wherein the tensioning mechanism applies a force to the cable to provide tension to the cable when the carriage is in a non-tensioning position. An activating device engages the carriage where manipulation of the activating device moves the carriage and provides tension to the cable which stiffens the adjustable arm.

### CROSS REFERENCE TO RELATED APPLICATIONS

The present application is continuation-in-part of U.S. patent application Ser. No. 10/684,764, filed on Oct. 14, 2003, which is a divisional of U.S. patent application Ser. No. 09/624,950, filed Jul. 25, 2000, which issued into U.S. Pat. No. 6,663,563, which is a continuation-in-part of U.S. patent application Ser. No. 09/409,294, filed Sep. 29, 1999, which issued into U.S. Pat. No. 6,210,325, which claims the benefit of U.S. provisional patent application Ser. No. 60/102,788, filed Oct. 2, 1998, the content of all of which are hereby incorporated by reference in their entirety.

### BACKGROUND OF THE INVENTION

The present invention generally relates to a retractor support apparatus that is useful in surgical procedures. More particularly, the present invention relates to an adjustable and flexible retractor support apparatus.

During many types of surgical procedures, such as micro-endoscopic and direct division discectomy, it is customary to use a retractor. The retractor is used to hold back tissue proximate a surgical incision to enable a surgeon to work at and in the surgical incision. Retractors typically include a blade and an arm, such as a shaft, to which the blade is attached. The retractor is generally held in place by attachment to a retractor support apparatus that is positioned over a support surface, such as an operating table. The retractor support is usually attached to a side rail located along one or more sides of the operating table by a clamping device, such as a fulcrum clamp or a cammed clamp.

During surgical procedures, it is preferable that the retractor be adjustable to precisely position the retractor in a selected position. Quickly securing the retractor in a selected position is also equally important in promoting efficient and safe surgical procedures. Simple horizontal and vertical adjustments of the retractor at the clamping device positioned along the sides of the operating table are also preferable for the surgical procedures.

Adjustable retractor support apparatuses, such as a gooseneck retractor support arm, provide a structure for precisely positioning a surgical device in a surgical site and securing the surgical device in the selected position. However, a cable in a gooseneck retractor support arm has a tendency to stretch over time with use. An elongated cable can cause the gooseneck retractor support arm to become unusable because the cable cannot be sufficiently tightened to secure the surgical device and the support arm in the selected position.

### SUMMARY OF THE INVENTION

The present invention includes a retractor system comprising an adjustable arm having a proximal end and a distal end where a cable is disposed through the adjustable arm from the proximal end to the distal end. A housing is attached to a proximal end of the adjustable arm and wherein a movable carriage is positioned within the housing. The carriage engages one end of the cable. A tensioning mechanism is positioned within the housing wherein the tensioning mechanism applies a force to the cable to provide tension to the cable when the carriage is in a non-tensioning position. An activating device engages and moves the carriage to a tensioning position to provide tension to the cable which stiffens the adjustable arm.

### BRIEF DESCRIPTION OF THE DRAWINGS

FIG. 1 is a perspective view of a surgical retractor apparatus of the present invention.

FIG. 2 is a side plan view of the cam-activated locking end of the surgical retractor apparatus of the present invention with portions shown in broken lines for clarity.

FIG. 3 is a sectional view of the cam-activated locking end of the surgical retractor apparatus of the present invention taken along Line 3-3 in FIG. 2.

FIG. 4 is an exploded perspective view of the cam-activated locking end of the surgical retractor apparatus of the present invention.

### DETAILED DESCRIPTION OF THE PREFERRED EMBODIMENTS

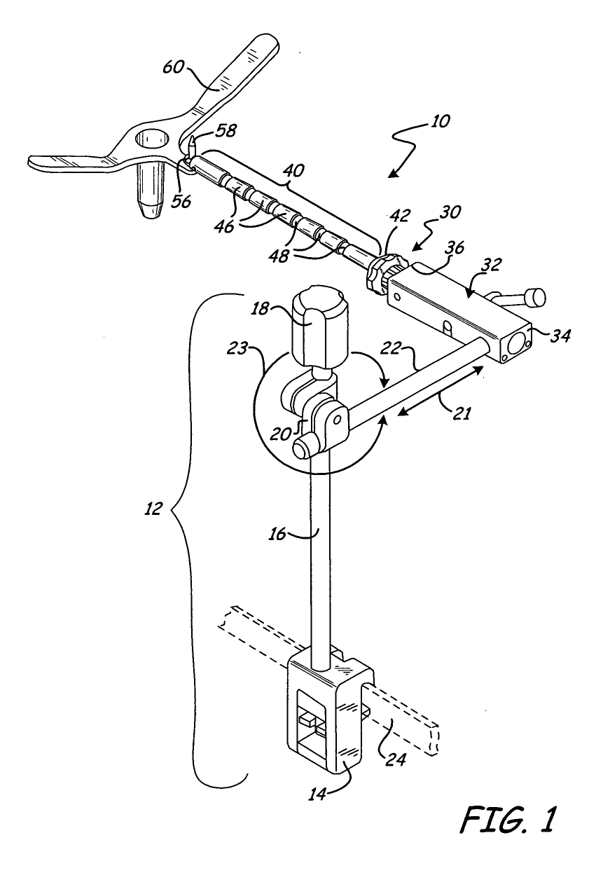

A retractor system of the present invention is generally illustrated at 10 in FIG. 1. The retractor system 10 includes a retractor support apparatus 30 attached to a clamping system 12.

The clamping system 12 includes a support clamp 14, a clamp support rod 16, a clamp knob 18, a retractor clamp 20, and a retractor support rod 22. A suitable clamping system 12, is described in U.S. Pat. No. 5,400,772 assigned to the same assignee as the present invention, and is incorporated herein by reference in its entirety. The clamping system 12 adjustably secures the retractor apparatus 30 to the clamp support rod 16 through the retractor clamp 20. The support clamp 14 clamps to a support surface, such as a rail 24 of an operating table (not shown).

Turning the clamp knob 18 releases the support clamp 14 and allows the clamping system 12 to be moved in a horizontal direction along the rail 24. Vertical adjustment of the clamping system 12 is accomplished by releasing the retractor clamp 20 and sliding the retractor clamp 20 along the clamp support rod 16 to a different vertical position. Horizontal adjustment of the retractor apparatus 30 occurs by releasing the retractor clamp 20 secured to the retractor support rod 22 and moving the retractor support rod 22 in a direction of arrows 21 if desired. Releasing the retractor clamp 20 also permits the retractor apparatus 30 to freely rotate 3600 about the clamp support rod 16, if desired, in the general direction indicated by arrows 23.

Referring to FIGS. 1, 3 and 4, the retractor support apparatus 30 includes an adjustable arm 40 operably connected to a clamping device 32 with a cable 50. The adjustable arm 40 is constructed of alternating cylinder(s) 46 and adjacent sphere(s) 48, connected to each other by the cable 50 extending centrally therethrough. The alternating cylinder(s) 46 and adjacent sphere(s) 48 form the adjustable arm 40 that is highly flexible and easy to manipulate in any direction by the surgeon. The adjustable arm 40 may be constructed of segments having a cylinder portion 46 and an adjacent sphere portion 48 that are integral with each other being machined from a single piece of metal where the segments are aligned to construct the adjustable arm 40.

Although alternating cylinders 46 and spheres 48 are used to practice the present invention, any pieced shape may be used to construct the adjustable arm 40, such as barrels, buckets, or the like. Other forms of pieced shapes may be chosen based upon the need to impart more or less flexibility to the adjustable arm 40.

The cable 50 is housed in a bore 52 (partially illustrated in FIG. 3) that extends through each cylinder 46 and adjacent sphere 48. A proximal end 54 of the cable 54 is positioned within a movable carriage 61 and includes a stopper 56 that engages a washer 64 having a cavity 66 that accepts the stopper 56. A distal end (not shown) of the cable 50 is attached to a docking mechanism 56 that accepts a peg 58 extending from a surgical device such as an access port 60 as illustrated in FIG. 1.

A plurality of spring washers 68 such as Belleville washers are positioned within a channel 70 in the carriage 61 wherein the spring washers 68 engage an end plate 63 of the carriage 62 and the washer 64 to bias the cable 50 into a tensioned state as illustrated in FIG. 3. Biasing or tensioning mechanisms other than a plurality of Belleville washers are within the scope of the present invention including, but not limited to, washers other than Belleville washers, such as a plurality of stacked urethane washers, and a compression spring.

The plurality of spring washers 68 compensate for the cable 50 being stretched or elongated over time with use. When the cable 50 is stretched or elongated, the clamping device 32 may not provide enough travel to compensate for the increase in length of the cable 50 and therefore may not be able to retain a surgical device such as the access port 60 within the docking mechanism 58 or retain the adjustable arm 40 in a selected position.

With the cable 50 under the selected tension, the clamping device 32 locks the adjustable arm 40 in the selected position. The clamping device 32 has a clamp support portion 34 and a distal end 36 as illustrated in FIG. 1. The distal end 36 receives a length adjusting end 42 of the adjustable arm 40.

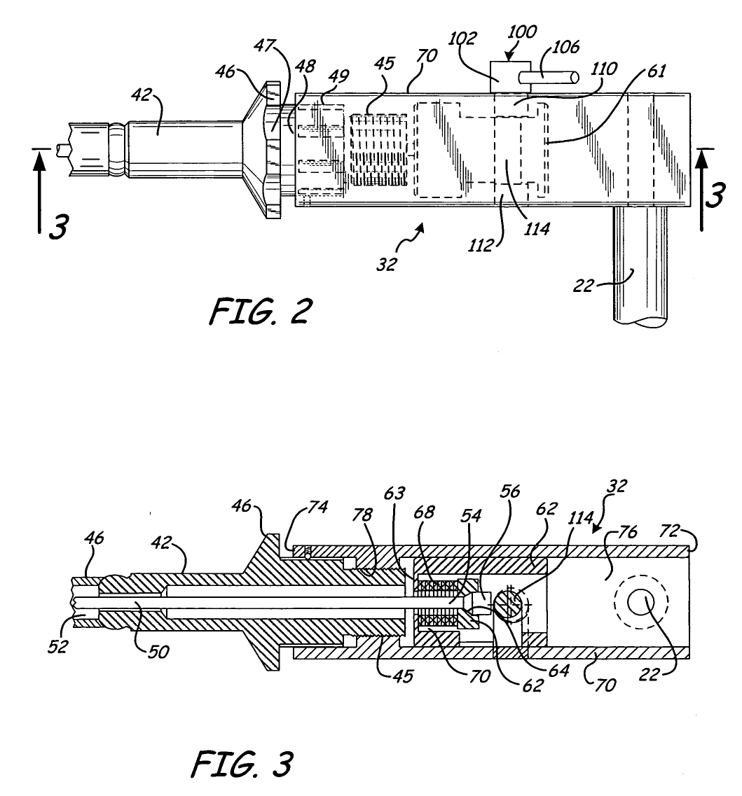

Referring to FIGS. 2-4, the clamping device 32 includes a housing 70 having a bore 76 that extends substantially centrally through the housing 70 from a proximal end 72 to a distal end 74. The length adjusting end 42 includes a threaded region 45 positioned intermediate the proximal end 72 and the distal end 74. The threaded region 45 threadably engages a threaded surface 78 of the through bore 76. The threaded region 45 is rotated by applying manual rotational force to a gripping surface 46 having plurality of indentions 47 where the gripping surface 46 is positioned proximate the distal end 74 of the housing 70.

Depending upon the rotational movement of the length adjusting end 42, the cable 50 becomes taut or slack. In a first rotational direction, the length adjusting end 42 is moved in a direction away from the clamping device 32 though the threaded engagement of the threaded region 45 with the threaded surface 78 which increases tension on the cable 50. In a second rotational direction, opposite the first rotational direction, the length adjusting device 42 moves toward the clamping device 32 by the threaded engagement of the threaded region 45 with the threaded surface 78 which decreases the tension on the cable 50.

The length adjusting end 42 is retained in a selected position due to an engagement of a spherical ball 80 that is positioned within an aperture 82 that intersects the cavity 76 with one of a plurality of channels 49 positioned about a perimeter of a cylindrical portion 48 of the length adjusting end 42. The plurality of channels 49 are substantially aligned with an axis 43 of the length adjusting end 42 and are substantially evenly spaced about the perimeter of the cylindrical portion 48. The spherical ball 80 is biased into the cavity 76 by a compression spring 84 that is retained within the aperture 82 with a threaded setscrew 86 engaging a threaded surface 83 defining the aperture 82.

With the cable 50 at a selected tension, one of the plurality of slots 49 aligns with the aperture 82 such that the spherical ball 80 is positioned within one of the slots 49. As the length adjusting end 42 is rotated, the plurality of slots 49 engage the spring loaded spherical ball 80 wherein the spherical ball 80 makes a clicking sound that alerts a surgeon or other healthcare provider that the length adjusting end 42 is secured in a selected position.

With the spherical ball 80 positioned within one of the slots 49, the length adjusting end 42 is retained in a selected position. Manual rotational force is required to overcome the bias of the compression spring 84 to reposition the length adjusting end 42 which increases or decreases the tension on the cable 50. Although a spherical ball 80 is a preferred retaining mechanism for engaging the plurality of slots 49, other configurations of the retaining mechanism are also within the scope of the present invention including, but not limited to, a spheroidal member and a cone.

Both the plurality of spring washers 80 and the threaded engagement of the length adjusting end 42 with the housing 70 allow for the adjustment of the tension of the cable 50. With the cable 50 adjusted to the proper tension, the carriage 61 moves within the housing 70 to position the cylinders 46 and adjacent spheres 48 of the adjusting arm 40 into either a relaxed or locked position.

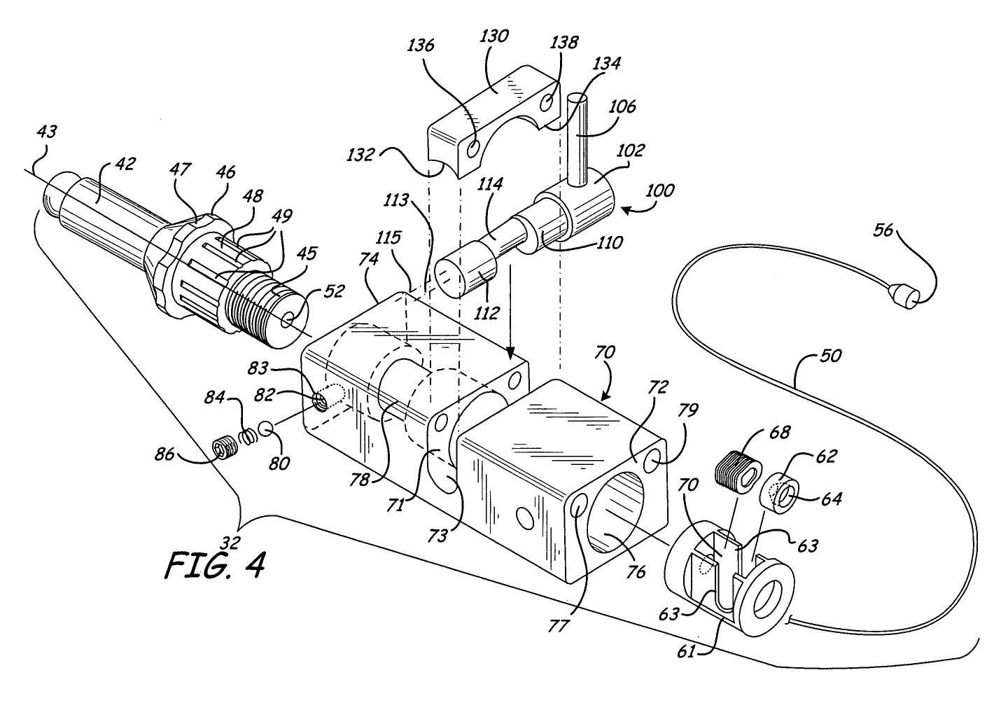

A camming structure 100 engages load bearing surfaces 63 on the carriage 61 to cause the carriage 61 to move and secure the adjusting arm 40 and the surgical device 60 in selected positions. The camming structure 100 has a cylindrical handle section 102 that attaches to a cam handle 106. Two cylindrical sections 110 and 112 of equal diameter are substantially positioned on a rotational axis 113. The two cylindrical sections 110 and 112 are preferably oriented substantially perpendicular to the cam handle 106 as illustrated in FIG. 4.

The camming action is provided by a camming section 114 preferably at an intermediate location between cylindrical sections 110 and 112. The camming section 114 is preferably smaller in diameter than the cylindrical sections 110 and 112. The camming section 114 has a cam axis 115 that is offset from the rotational axis 113. When the cam handle 106 on the cylindrical handle section 102 is rotated about the rotational axis 113, the cam section 114 engages the load bearing surfaces 63 and thereby moves the carriage 61 to place tension on the cable 50 or remove tension from the cable 50.

The camming structure 100 also engages the slot 71 in the housing 70. The slot 71 has lower semi-cylindrical bottom surfaces, one of which is shown at 73, that accepts cylindrical sections 110 and 112. A cap 130 fits into the slot 71 of the housing 70 and secures the camming structure 100 therein while permitting the camming structure 100 to rotate about the axis 113. The cap 130 engages the cylindrical sections 110 and 112 with arcuate downwardly facing surfaces 132 and 134, respectively.

When the cap 130 is positioned within slot 71, apertures 136 and 138 are aligned with apertures 77 and 79, respectively. Cap screws (not shown) are inserted into apertures 77 and 79 and extend into apertures 136 and 138 of the cap 130. The cap 130 also prevents the camming structure 100 from moving upward or downward when disposed in slot 71.

Although the present invention has been described with reference to preferred embodiments workers skilled in the art would recognize that changes maybe made in form and detail without departing from the spirit and scope of the invention.

What is claimed is:

1. A retractor system comprising:
an adjustable arm having a proximal end and a distal end;
a cable disposed through the adjustable arm from the proximal end to the distal end;
a housing attached to the proximal end of the adjustable arm;
a carriage disposed within the housing wherein the carriage engages one end of the cable and wherein the carriage moves within the housing;
a tensioning mechanism disposed within the housing and wherein the tensioning mechanism applies a force to the cable to provide tension to the cable; and
an activating device engages the carriage wherein manipulation of the activating device moves the carriage and provides tension on the cable which stiffens the adjustable arm.

2. The retractor system of claim 1 and wherein the tensioning mechanism comprises a plurality of stacked spring washers disposed about the cable.

3. The retractor system of claim 1 and wherein the tensioning mechanism comprises a plurality of Belleville washers.

4. The retractor system of claim 1 and wherein the tensioning mechanism comprises a compression spring.

5. The retractor system of claim 1 and wherein the tensioning mechanism comprises a plurality of urethane washers.

6. The retractor system of claim 1 and wherein the activating device comprises a camming surface wherein the camming surface rotates such that the carriage moves to place tension upon the cable and stiffen the adjustable arm.

7. The retractor system of claim 1 and wherein the adjustable arm comprises alternating balls and cylinders connected to each other by the cable extending centrally therethrough.

8. The retractor system of claim 1 and further comprising an engagement mechanism attached to another end of the cable and wherein the engagement mechanism engages a distal end of the adjustable arm and wherein when the activating device moves to create tension upon the cable, the engagement mechanism moves to secure a surgical device thereto.

9. A retractor system comprising:
an adjustable arm having a length adjusting end and an engaging end;
a cable positioned within the adjustable arm;
a housing operably attached to the length adjusting end and having a cavity for accepting the length adjusting end wherein the length adjusting end includes a cylindrical portion having a plurality of axially aligned slots; and
at least one spring loaded retaining mechanism at least partially disposed within the cavity and engaging the plurality of axially aligned grooves wherein as the length engaging end is rotated into a selected position, tension is increased or decreased on the cable and wherein the retaining mechanism engages one of the plurality of slots to secure the length adjusting end in the selected position.

10. The retractor system of claim 9 and wherein the operable attachment between the length adjusting end and the housing comprises a threaded engagement.

11. The retractor system of claim 9 and wherein the length adjusting end further comprises a gripping surface external to the housing and wherein manual force applied to the gripping surface rotates the length adjusting end and adjusts the tension on the cable.

12. The retractor system of claim 9 and further comprising:
a carriage positioned within the housing and having an end of the cable attached thereto; and
a camming mechanism operably engaging the carriage wherein the camming mechanism rotates to move the carriage such that the adjustable arm stiffens in a selected positions.

13. A retractor system mounted to a surgical table comprising:
a post attached to the surgical table and extending upwardly therefrom;
a support arm attached to the post and extending over the table;
a housing attached to the support arm;
a carriage disposed within the housing and movable therein;
an adjustable arm operably attached to the carriage and having a cable disposed therethrough wherein one end of the cable engages the carriage and another end engages an engagement mechanism for retaining a surgical device in a selected position;
an activating device operably engaging the carriage wherein manipulation of the activating device moves the carriage to place tension upon the cable and secure the adjustable arm and the engagement end in selected positions; and
a tensioning mechanism positioned about the cable within the housing to retain tension on the cable when the carriage positions into a non-tensioning position.

14. The retractor system of claim 13 and wherein the tensioning mechanism comprises a plurality of spring washers.

15. The retractor system of claim 13 and wherein the tensioning mechanism comprises a plurality of Belleville washers.

16. The retractor system of claim 13 and wherein the tensioning mechanism comprises a plurality of urethane washers.

17. The tensioning mechanism of claim 13 and wherein the tensioning mechanism comprises a compression spring.

18. The retractor system of claim 13 and wherein the activating device comprises a camming member and wherein the camming member rotates and engages the carriage such that the adjustable arm and engagement end are secured in selected positions.

19. The retractor system of claim 13 wherein the adjustable arm comprises:
a length adjusting end comprising:
a cylindrical portion having a plurality of grooves therein; and
a threaded portion and wherein the threaded portion engages a threaded surface in the housing to operably move the length adjusting end;

20. The retraction system of claim 19 and wherein the housing comprises an aperture having a spring loaded retaining mechanism at least partially positioned therein and wherein the spring loaded retaining mechanism engages one of the plurality of slots or channels such that the length adjusting end retains in a selected position.
